(19) United States
(12) Patent Application Publication
(10) Pub. No.: US 2026/0245212 A1
(43) Pub. Date: Aug. 20, 2026

(54) IMAGE PROCESSING DEVICE, IMAGE PROCESSING METHOD, AND STORAGE MEDIUM

(71) Applicant: NEC Corporation, Tokyo (JP)

(72) Inventors: Yoshifumi ONISHI, Tokyo (JP); Terumi UMEMATSU, Tokyo (JP)

(73) Assignee: NEC Corporation, Tokyo (JP)

(21) Appl. No.: 19/161,513

(22) PCT Filed: Mar. 13, 2023
(86) PCT No.: PCT/JP2023/009549

§ 371 Date: Sep. 2, 2025

(51) Int. Cl. G06T 7/00; A61B 3/11; G06T 7/60; G06T 7/73; G16H 10/60

(52) U.S. Cl. CPC G06T 7/0016; A61B 3/111; A61B 3/112; G06T 7/60; G06T 7/73; G16H 10/60; G06T 2207/10016; G06T 2207/30041; G06T 2207/30201

(57) ABSTRACT

An image processing device 1X mainly includes a convergence measurement means 15X, a determination means 16X, and a pupil measurement means 17X. The convergence measurement means 15X is configured to measure a degree of convergence of a subject after eye opening, based on an image obtained by capturing the subject who has performed the eye opening after eye closing. The determination means 16X is configured to determine presence or absence of a focus change of the subject, based on the degree of convergence. The pupil measurement means 17X is configured to measure a pupil of the subject based on the image obtained by capturing the subject, upon determining the absence of the focus change.

### TECHNICAL FIELD

The present disclosure relates to a technical field of an image processing device, an image processing method, and a storage medium that perform processing related to measurement of a light reflex using an image.

### BACKGROUND

A system that measures a pupil of a subject based on a facial image obtained by capturing the subject has been known. For example, PTL 1 discloses a system that detects a region of a pupil of a subject based on video signals from two provided cameras and carries out analysis and the like regarding a pupil area, a pupil diameter, and a pupil position.

### CITATION LIST

### Patent Literature

PTL 1: WO 2002/003853 A1

### SUMMARY

### Problem to be Solved

A dedicated device or system has been generally required to measure a light reflex. Meanwhile, in a case of routinely measuring a light reflex for the purpose of self-care or the like, it is desirable that the subject him/herself is enabled to easily carry out the measurement.

In view of the problem described above, an object of the present disclosure is to provide an image processing device, an image processing method, and a storage medium capable of suitably generating information related to a light reflex of a subject from an image obtained by capturing the subject.

### Means For Solving the Problem

One mode of the health information processing device is a health information processing device including:
a convergence measurement means for measuring a degree of convergence of a subject after eye opening, based on an image obtained by capturing the subject who has performed the eye opening after eye closing;
a determination means for determining presence or absence of a focus change of the subject, based on the degree of convergence; and a pupil measurement means for measuring a pupil of the subject based on the image obtained by capturing the subject, upon determining the absence of the focus change.

One mode of the image processing method is an image processing method executed by a computer, includes:
measuring a degree of convergence of a subject after eye opening, based on an image obtained by capturing the subject who has performed the eye opening after eye closing;
determining presence or absence of a focus change of the subject, based on the degree of convergence; and
measuring a pupil of the subject based on the image obtained by capturing the subject, upon determining the absence of the focus change.

One mode of the storage medium is a storage medium storing a program executed by a computer, the program causing the computer to:
measure a degree of convergence of a subject after eye opening, based on an image obtained by capturing the subject who has performed the eye opening after eye closing;
determine presence or absence of a focus change of the subject, based on the degree of convergence; and
measure a pupil of the subject based on the image obtained by capturing the subject, upon determining the absence of the focus change.

### EFFECT

An example advantage according to the present invention is to suitably generate a summary of medical treatment records in a target period.

### BRIEF DESCRIPTION OF THE DRAWINGS

FIG. 1 illustrates a schematic configuration of a light reflex measurement system according to a first example embodiment.

FIG. 2 illustrates a state when a light reflex is measured in a case where the light reflex measurement system is a single terminal device.

FIG. 3 illustrates an exemplary hardware configuration of an image processing device common to example embodiments.

FIG. 4 is exemplary functional blocks of an image processing device related to a light reflex measurement process according to the first example embodiment.

FIG. 5 is an exemplary camera image obtained by capturing a face of a subject.

FIG. 6 is an exemplary graph illustrating a temporal change in a pupil size with the light reflex.

FIG. 7 is an exemplary flowchart regarding the light reflex measurement process according to the first example embodiment.

FIG. 8 illustrates a schematic configuration of a light reflex measurement system according to a modified example.

FIG. 9 illustrates a schematic configuration of a light reflex measurement system according to a second example embodiment.

FIG. 10 is a block diagram of an image processing device according to a third example embodiment.

FIG. 11 is an exemplary flowchart to be executed by the image processing device according to the third example embodiment.

### Example Embodiment

Hereinafter, example embodiments of an image processing device, an image processing method, and a storage medium will be described with reference to the drawings.

### FIRST EXAMPLE EMBODIMENT

### (1) System Configuration

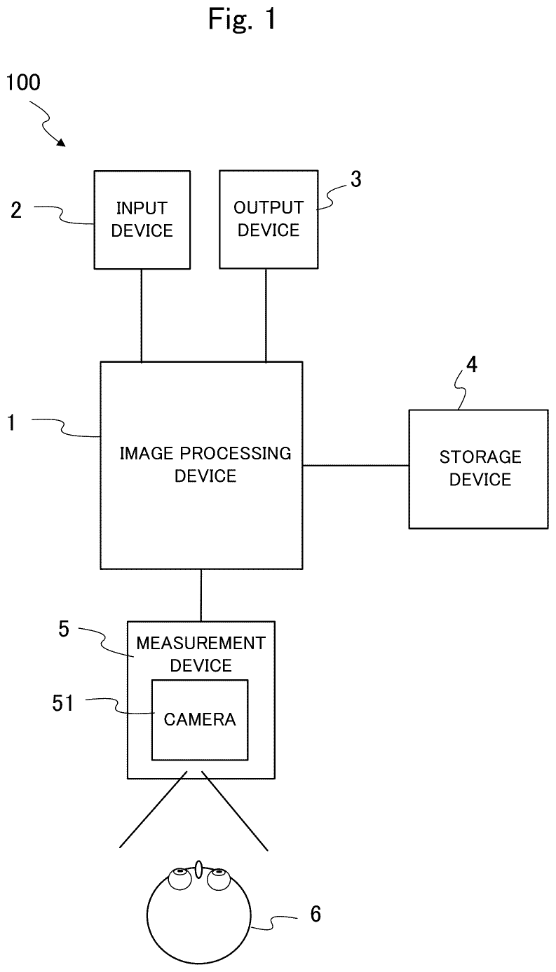

FIG. 1 illustrates a schematic configuration of a light reflex measurement system 100 according to a first example embodiment. The light reflex measurement system 100 is a system that simply measures a light reflex of a subject 6 based on an image obtained by capturing the face of the subject 6 with a visible light camera, and mainly includes an image processing device 1, an input device 2, an output device 3, a storage device 4, and a measurement device 5 including a camera (imaging device) 51. For example, the subject 6 simply measures his/her own light reflex using the light reflex measurement system 100 for the purpose of managing a health condition (including self-care).

The image processing device 1 measures the light reflex of the subject 6 based on a facial image (including video, which is a sequence of a predetermined number of images obtained in time series; the same applies hereinafter) of the subject 6 generated by the camera 51, and outputs a measurement result. The image processing device 1 performs data communication with the input device 2, the output device 3, the storage device 4, and the measurement device 5 via a communication network or wired or wireless direct communication.

The input device 2 is an interface that receives a user input (manual input). A user who inputs information using the input device 2 may be the subject 6 him/herself, or may be a person who manages or supervises the subject 6. The input device 2 may be, for example, various types of user input interfaces, such as a touch panel, a button, a keyboard, a mouse, an audio input device, and the like. The input device 2 supplies an input signal generated based on the user input to the image processing device 1.

The output device 3 outputs predetermined information based on an output signal supplied from the image processing device 1. In this case, the output signal includes at least one of a display signal and an audio signal. Then, the output device 3 displays information based on the display signal supplied from the image processing device 1, and outputs information by sound based on the audio signal supplied from the image processing device 1. The output device 3 includes, for example, at least one of a display device, such as a display or a projector, and an audio output device, such as a speaker.

The storage device 4 is a memory that stores various types of information required to measure the light reflex, for example. The storage device 4 may be an external storage device, such as a hard disk connected to or incorporated in the image processing device 1, or may be a storage medium, such as a flash memory. The storage device 4 may be a server device that performs data communication with the image processing device 1. The storage device 4 may include a plurality of devices.

The measurement device 5 is one or a plurality of sensors including the camera 51, which is a visible light camera. For example, the measurement device 5 may include an illuminance sensor for detecting a change in a light amount under a measurement environment of the light reflex of the subject 6. The measurement device 5 supplies signals measured by the sensors to the image processing device 1. Hereinafter, the image generated by the camera 51 will also be referred to as a “camera image”. The camera 51 is an example of an “imaging means”.

The configuration of the light reflex measurement system 100 illustrated in FIG. 1 is an example, and various modifications may be made to the configuration. For example, the image processing device 1, the input device 2, the output device 3, the storage device 4, and the measurement device 5 may be implemented by a single terminal device as a smartphone, a tablet terminal, or the like.

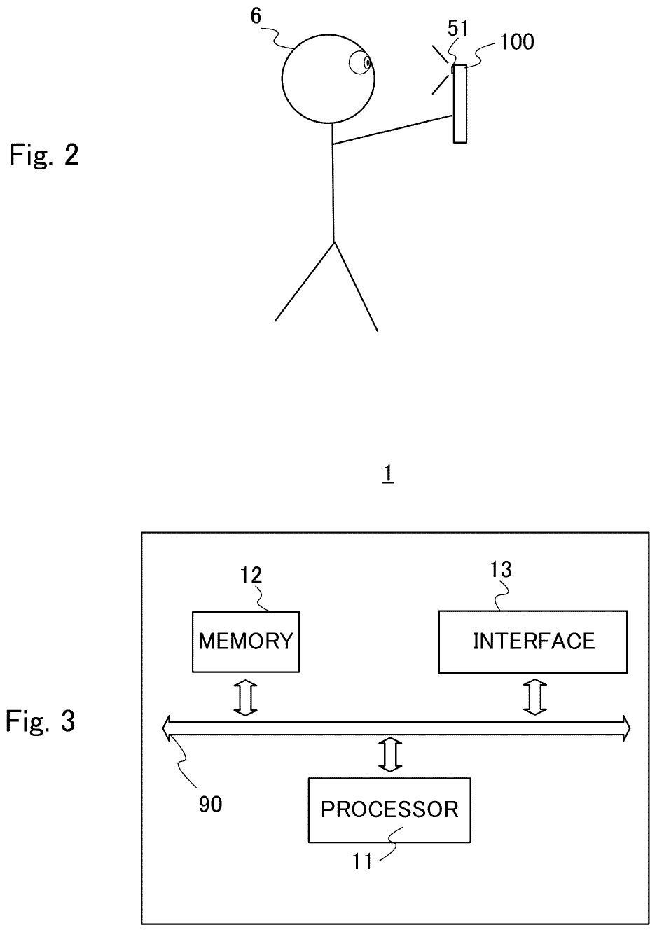

FIG. 2 illustrates a state when the light reflex is measured in a case where the light reflex measurement system 100 is a single terminal device (e.g., smartphone). As illustrated in FIG. 2, the subject 6 holds the light reflex measurement system 100, which is a terminal device, and adjusts the orientation of the terminal device in such a way that his/her own face is included in an imaging range of the camera 51. The light reflex measurement system 100 may be fixed to a tripod or the like. Then, in the state illustrated in FIG. 2, the subject 6 closes eyes for a predetermined time and then opens the eyes in accordance with an instruction (guidance) output by the light reflex measurement system 100, and causes the light reflex measurement system 100 to measure the light reflex.

### (2) Hardware Configuration

FIG. 3 illustrates a hardware configuration of the image processing device 1. The image processing device 1 includes, as hardware, a processor 11, a memory 12, and an interface 13. The processor 11, the memory 12, and the interface 13 are coupled to each other via a data bus 90.

The processor 11 functions as a controller (arithmetic device) that takes overall control of the image processing device 1 by executing a program stored in the memory 12. The processor 11 is, for example, a processor such as a central processing unit (CPU), a graphics processing unit (GPU), a tensor processing unit (TPU), or the like. The processor 11 may include a plurality of processors. The processor 11 is an example of a computer.

The memory 12 includes various volatile memories and nonvolatile memories, such as a random access memory (RAM), a read only memory (ROM), a flash memory, and the like. The memory 12 stores a program for executing processing to be performed by the image processing device 1. A part of information stored in the memory 12 may be stored in one or a plurality of external storage devices capable of communicating with the image processing device 1, or may be stored in a storage medium detachable from the image processing device 1. The memory 12 may function as at least a part of the storage device 4.

The interface 13 is an interface for electrically connecting the image processing device 1 with another device. Those interfaces may be a wireless interface such as a network adapter for wirelessly exchanging data with the another device, or may be a hardware interface for connecting to the another device by a cable or the like.

The hardware configuration of the image processing device 1 is not limited to the configuration illustrated in FIG. 3. For example, the image processing device 1 may include at least one of the input device 2, the output device 3, the storage device 4, and the measurement device 5.

### (3) Outline of Light Reflex Measurement Process

Next, a light reflex measurement process, which is a process related to measurement of a light reflex, will be described. In outline, the image processing device 1 sequentially instructs the subject 6 to close the eyes and open the eyes, and measures a pupil size of the subject 6 in time series based on the camera image generated after the opening of the eyes. At this time, the image processing device 1 measures a degree of convergence to determine whether there is a focus change in the pupil size measurement period, and uses the pupil size measurement result when there is no focus change to calculate an index of the light reflex. As a result, the image processing device 1 is enabled to accurately measure the light reflex based on the camera image.

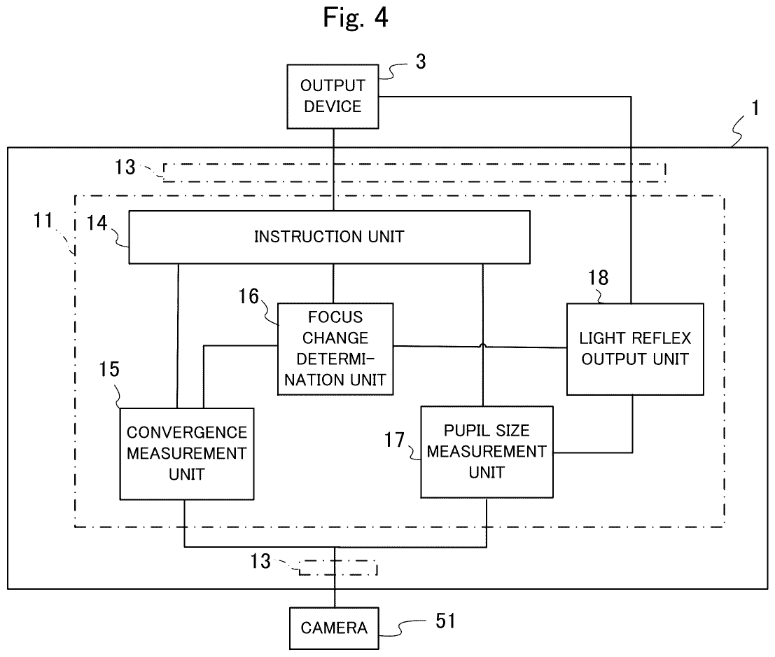

FIG. 4 is exemplary functional blocks of the image processing device 1 related to the light reflex measurement process. The processor 11 of the image processing device 1 functionally includes an instruction unit 14, a convergence measurement unit 15, a focus change determination unit 16, a pupil size measurement unit 17, and a light reflex output unit 18. While blocks between which data is exchanged are connected by a solid line in FIG. 4, combinations of the blocks between which the data is exchanged are not limited to those illustrated in the drawing. Other functional block diagrams to be described later are in a similar manner.

When the user input through the input device 2 instructing the start of the light reflex measurement is detected, or when the light reflex measurement timing planned in advance is reached, the instruction unit 14 determines that the light reflex measurement is to be started, and sequentially instructs the subject 6 to close the eyes and to open the eyes. In that case, the instruction unit 14 issues an instruction regarding the opening of the eyes after a predetermined time elapses after the instruction regarding the closing of the eyes. The time length of the predetermined time described above is, for example, a time length of the closing of the eyes required to observe a light reflex, and a preset value is stored in the storage device 4, the memory 12, or the like. In that case, the instruction unit 14 may detect the closing of the eyes of the subject 6 from the camera image using any face recognition technique. In that case, the instruction unit 14 issues an instruction regarding the opening of the eyes after a predetermined time elapses after the detection of the closing of the eyes of the subject 6 from the camera image.

The instruction unit 14 suitably issues an instruction (which will also be referred to as a “pre-closing line-of-sight instruction”) for the subject 6 to look at (i.e., focus on) a location a predetermined distance away to stabilize the focus of the subject 6 before the instruction regarding the closing and opening of the eyes. The “predetermined distance” described above is, for example, a distance of equal to or more than 6 m at which the focal length is substantially infinite. The location the predetermined distance away is not limited to the location equal to or more than 6 m away, and may be any location at which the distance to the subject 6 does not change. Then, when the focus change determination unit 16 determines that there is no focus change after the issuance of the pre-closing line-of-sight instruction, the instruction unit 14 sequentially issues the instructions regarding the closing and opening of the eyes. The instruction unit 14 notifies the convergence measurement unit 15, the focus change determination unit 16, and the pupil size measurement unit 17 of the issuance of the instruction regarding the closing and opening of the eyes, and notifies the convergence measurement unit 15 and the focus change determination unit 16 of the issuance of the pre-closing line-of-sight instruction.

The convergence measurement unit 15 measures a level of convergence (i.e., cross-eyed state) of the eyes of the subject 6 (which will also be referred to as a “degree of convergence”) based on the camera images obtained from the camera 51 via the interface 13. Here, the convergence measurement unit 15 measures the degree of convergence based on the camera images generated in a period during which the light reflex of the subject 6 is estimated to occur (which will also be referred to as a “light reflex estimation period”). The target period is determined to, for example, a period until a predetermined time elapses after opening of the eyes of the subject 6 in response to an eye opening instruction issued by the instruction unit 14. The predetermined time described above is a time length in which the light reflex is estimated to occur after the eye opening, and a preset value is stored in the storage device 4, the memory 12, or the like. In that case, the convergence measurement unit 15 detects, using any image recognition technique, the eye opening of the subject 6 based on the latest camera image obtained from the camera 51 after the eye opening instruction issued by the instruction unit 14, and recognizes, as the light reflex estimation period, the period until the predetermined time elapses from the time at which the eye opening of the subject 6 is detected. The light reflex estimation period is an example of a “predetermined period”.

When the pre-closing line-of-sight instruction is issued, the convergence measurement unit 15 measures a degree of convergence based on the camera images obtained after the pre-closing line-of-sight instruction. In that case, for example, the convergence measurement unit 15 measures the degree of convergence in a period from the pre-closing line-of-sight instruction to the eye closing instruction. The convergence measurement unit 15 supplies the measured degree of convergence to the focus change determination unit 16.

The focus change determination unit 16 determines whether there is a focus change of the subject 6 in the light reflex estimation period, and determines, based on a result of the determination, whether measurement and output of the light reflex by the light reflex output unit 18 is required. That is, the focus change determination unit 16 determines that the stability (i.e., stationarity) of the focus of the subject 6 in the light reflex estimation period is a necessary condition for measuring and outputting a light reflex. Thus, only when the focus of the subject 6 is stable in the light reflex estimation period, the focus change determination unit 16 instructs the light reflex output unit 18 to measure (i.e., calculate a light reflex index to be described later) and output the light reflex.

Here, in general, presence or absence of the focus change may be determined by presence or absence of a change in the degree of convergence, and it may be considered that there is no focus change when there is no change in the degree of convergence. Thus, the focus change determination unit 16 determines the presence or absence of the focus change of the subject 6 in the light reflex estimation period based on the time-series degree of convergence measured by the convergence measurement unit 15 from the camera images generated in the light reflex estimation period. For example, the focus change determination unit 16 determines whether the degree of convergence substantially changes based on a variance of a predetermined number of degrees of convergence obtained in the light reflex estimation period, a difference between a maximum value and a minimum value, or comparison between a predetermined threshold and statistics representing other variations. The threshold described above is stored in advance in the storage device 4, the memory 12, or the like, for example.

Then, when the degree of convergence is determined to be substantially changed, the focus change determination unit 16 determines that a focus change has occurred in the light reflex estimation period. Then, in that case, the focus change determination unit 16 requests the instruction unit 14 to re-output the instruction. Here, in the camera images obtained in the light reflex estimation period in which the focus change has occurred, it is highly likely that a pupil change caused by the focus change, which is known as a near response, has occurred and the light reflex may not be accurately measured. Thus, when it is determined that the focus change has occurred in the light reflex estimation period, the focus change determination unit 16 requests the instruction unit 14 to issue an instruction for re-measurement of the light reflex. The instruction unit 14 sequentially outputs the pre-closing line-of-sight instruction and the instructions regarding the closing and opening of the eyes when the pre-closing line-of-sight instruction has been issued, and sequentially outputs the instructions regarding the closing and opening of the eyes when the pre-closing line-of-sight instruction has not been issued.

On the other hand, when the degree of convergence is determined not to be substantially changed, the focus change determination unit 16 determines that no focus change has occurred in the light reflex estimation period. Then, in that case, the focus change determination unit 16 instructs the light reflex output unit 18 to measure and output a light reflex. Here, when no focus change has occurred in the light reflex estimation period, there is no pupil change caused by the focus change, and the light reflex of the subject 6 may be accurately measured from the camera images obtained in the light reflex estimation period. Therefore, the light reflex output unit 18 may output an accurate measurement result of the light reflex by measuring and outputting the light reflex when it is determined that no focus change has occurred in the light reflex estimation period.

It is suitable that the focus change determination unit 16 may further determine whether there is a focus change of the subject 6 before the eye closing and after the eye opening of the subject 6 in addition to the determination as to whether there is a focus change of the subject 6 in the light reflex estimation period. That is, the focus change determination unit 16 may set, as a condition (necessary condition) for measuring and outputting the light reflex, the stability of the focus of the subject 6 before the eye closing based on the eye closing instruction and after the eye opening based on the eye opening instruction. In that case, the focus change determination unit 16 determines the presence or absence of the focus change of the subject 6 described above based on, for example, a difference between the degree of convergence measured by the convergence measurement unit 15 immediately before the eye closing of the subject 6 and the degree of convergence measured by the convergence measurement unit 15 after the eye opening of the subject 6. Then, when the difference described above is larger than a threshold, the focus change determination unit 16 determines that the focus change of the subject 6 has occurred before and after the eye closing and eye opening, and requests the instruction unit 14 to re-output the instruction regardless of the presence or absence of the focus change in the light reflex estimation period. On the other hand, when there is no focus change of the subject 6 before the eye closing and after the eye opening and there is no focus change in the light reflex estimation period, the focus change determination unit 16 instructs the light reflex output unit 18 to measure and output the light reflex. Even when there is no focus change of the subject 6 before the eye closing and after the eye opening, the focus change determination unit 16 requests the instruction unit 14 to re-output the instruction if there is a focus change in the light reflex estimation period. As described above, by setting the stability of the focus before and after the instructions regarding the eye closing and eye opening as a necessary condition for measuring the light reflex, it becomes possible to obtain more accurate measurement result of the light reflex.

When the pre-closing line-of-sight instruction has been issued, the focus change determination unit 16 may set the stability of the focus of the subject 6 after the pre-closing line-of-sight instruction as a necessary condition for issuing the eye closing instruction, and may determine the presence or absence of the focus change of the subject 6 after the pre-closing line-of-sight instruction. In that case, for example, the focus change determination unit 16 determines the presence or absence of a change in the time-series degree of convergence supplied from the convergence measurement unit 15 in the immediately preceding predetermined time. In that case, for example, the focus change determination unit 16 determines whether the degree of convergence substantially changes in the predetermined time described above based on a variance of a predetermined number of degrees of convergence obtained in the predetermined time, a difference between a maximum value and a minimum value, or comparison between a predetermined threshold and statistics representing other variations. Then, when the degree of convergence is determined not to be substantially changed, the focus change determination unit 16 determines that there is no focus change of the subject 6 (i.e., focus is stable), and instructs the instruction unit 14 to output an instruction regarding closing of the eyes. On the other hand, when the degree of convergence is determined to be substantially changed, the focus change determination unit 16 determines that there is a focus change of the subject 6 (i.e., focus is not stable), and continues to determine the presence or absence of a change in the time-series degree of convergence supplied in the immediately preceding predetermined time.

The pupil size measurement unit 17 measures a pupil size of the subject 6 in the light reflex estimation period. In this case, the pupil size measurement unit 17 measures the pupil size of the subject 6 from each of the camera images generated in the light reflex estimation period, thereby generating a measurement result of the time-series pupil size of the subject 6 in the light reflex estimation period. Then, the pupil size measurement unit 17 supplies the measurement result of the pupil size to the light reflex output unit 18.

The pupil size measurement unit 17 may measure the pupil size of the subject 6 from each of the camera images generated in the light reflex estimation period regardless of the result of the determination made by the focus change determination unit 16 regarding the presence or absence of the focus change of the subject 6. In this case, for example, the pupil size measurement unit 17 may output, to the light reflex output unit 18, the measurement result of the pupil size generated in the light reflex estimation period only when the determination result indicating that no focus change has occurred is received from the focus change determination unit 16. As another example, the pupil size measurement unit 17 may receive the determination result regarding the presence or absence of the focus change by the focus change determination unit 16, and may output, to the light reflex output unit 18, the determination result and the measurement result of the pupil size generated in the light reflex estimation period.

Instead of the example described above, the pupil size measurement unit 17 may measure the pupil size of the subject 6 from each of the camera images generated in the light reflex estimation period and accumulated in the storage device 4, the memory 12, or the like after the focus change determination unit 16 determines that there is no focus change of the subject 6. That is, in that case, when the focus change determination unit 16 determines that there is a focus change of the subject 6, the pupil size measurement unit 17 does not measure the pupil size based on the camera images generated in the target light reflex estimation period.

The light reflex output unit 18 calculates an index regarding the light reflex (which will also be referred to as a “light reflex index”) based on the measurement result of the pupil size by the pupil size measurement unit 17 in the light reflex estimation period, and displays or audio-outputs information regarding a result of the calculation of the light reflex index using the output device 3. Here, the light reflex output unit 18 carries out the calculation of the light reflex index or the like when an instruction regarding measurement and output of the light reflex is issued from the focus change determination unit 16, that is, when it is determined that no focus change of the subject 6 has occurred in the light reflex estimation period (and before the eye closing and after the eye opening). A type of the light reflex index to be calculated differs depending on an application to which the light reflex measurement system 100 is applied, and specific examples thereof will be described later in sections of “(4) Degree of Convergence, Pupil Size, and Light Reflex Index” and “(7) Application”.

Here, the information regarding the result of the calculation of the light reflex index caused to be output from the output device 3 by the light reflex output unit 18 may be information indicating the calculated light reflex index itself, or may be information regarding a state or the like of the subject 6 estimated based on the light reflex index. An example of the latter case will be specifically described in the section of “(7) Application”. The light reflex output unit 18 generates an output signal (i.e., at least one of a display signal and an audio signal) for outputting the information regarding the calculation result of the calculated light reflex index, and supplies the output signal to the output device 3 via the interface 13. As a result, the light reflex output unit 18 causes the output device 3 to output the information regarding the calculation result of the light reflex index.

The image processing device 1 may calculate the light reflex index for a predetermined number of times by executing the processing of the instruction unit 14, the convergence measurement unit 15, the focus change determination unit 16, the pupil size measurement unit 17, and the light reflex output unit 18 a predetermined number of times. In that case, the light reflex output unit 18 calculates a time average or another representative value of the light reflex index for the predetermined number of times, and causes the output device 3 to output a result of the calculation. As a result, a highly accurate light reflex index subjected to the statistical processing may be output.

The components of the instruction unit 14, the convergence measurement unit 15, the focus change determination unit 16, the pupil size measurement unit 17, and the light reflex output unit 18 described with reference to FIG. 4 may be implemented by, for example, the processor 11 executing a program. Each component may be implemented by a required program being recorded in any nonvolatile storage medium and installed as appropriate. At least some of those components may not be limited to be implemented by software based on a program, and may be implemented by, for example, a combination of any of hardware, firmware, and software. At least some of those components may be implemented using, for example, a user-programmable integrated circuit, such as a field-programmable gate array (FPGA), a microcontroller, or the like. In that case, a program including the components described above may be implemented using the integrated circuit. At least some of the components may include an application specific standard produce (ASSP), an application specific integrated circuit (ASIC), or a quantum processor (quantum computer control chip). As described above, the components may be implemented by various types of hardware. Other example embodiments to be described later are in a similar manner to the descriptions above. Moreover, those components may be implemented by, for example, a plurality of computers in cooperation with each other using cloud computing technology or the like.

### (4) Degree of Convergence, Pupil Size, and Light Reflex Index

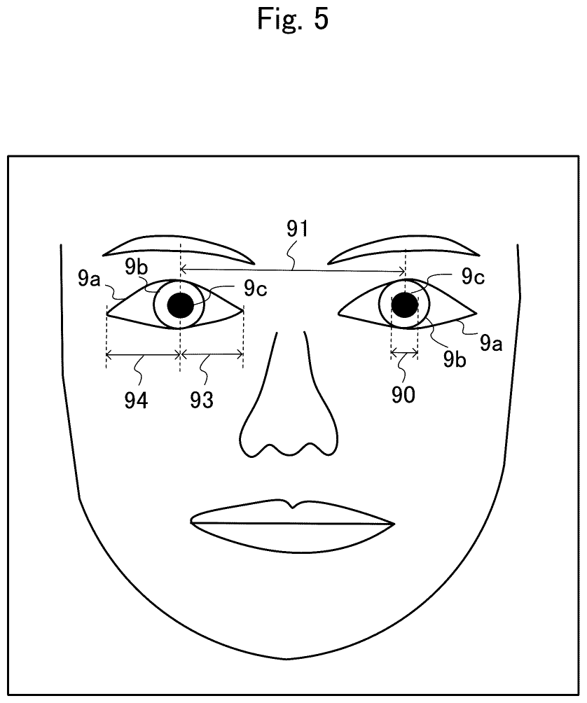

First, specific examples of the degree of convergence and the pupil size will be described. FIG. 5 is an exemplary camera image obtained by capturing the face of the subject 6.

The image processing device 1 recognizes, based on any image recognition technique, each area of an eye (both eyes in this case) 9a, an iris 9b, and a pupil 9c of the subject 6 from a camera image obtained by capturing the face of the subject 6. Then, the image processing device 1 calculates a pupil size and a degree of convergence based on the recognized areas.

First, calculation of the pupil size will be described. The image processing device 1 calculates a pupil size (i.e., pupil diameter) equivalent to a length of an arrow 90. Here, the image processing device 1 may calculate a pupil size of each of both eyes, and may further generate an average of the calculated pupil sizes.

Next, calculation of the degree of convergence will be described. The image processing device 1 calculates an interpupillary distance equivalent to a length of an arrow 91 as an example of the degree of convergence. In this manner, by calculating the degree of convergence based on both eyes, the image processing device 1 is enabled to recognize accurate convergence. In that case, the camera image is an image obtained by capturing at least both eyes of the subject 6, and the image processing device 1 calculates the degree of convergence based on the positions of the pupils 9c in the camera image.

The image processing device 1 may calculate pupil information based on a camera image obtained by capturing only one eye of the subject. In that case, for example, the image processing device 1 may calculate, as a degree of convergence, a relative position (i.e., distance equivalent to lengths of arrows 93 and 94) of the center of the pupil 9c with respect to both ends of the eye 9a. In this manner, the image processing device 1 may calculate, as a degree of convergence, a value indicating a relative position of the pupil 9c in the eye 9a.

Here, the image processing device 1 may perform normalization processing of converting the pupil size, the interpupillary distance, and the like from a size based on the number of pixels in the camera image into a size of a predetermined scale (e.g., actual size), and may generate a normalized pupil size and interpupillary distance. In that case, for example, the image processing device 1 may normalize the pupil size and the interpupillary distance based on the size of the iris 9b by using characteristics that the size of the iris 9b has little individual difference and does not change over time. In that case, for example, information indicating a relationship between the size of the iris 9b in the image and the size of the iris 9b after the normalization is stored in advance in the storage device 4, the memory 12, or the like, and the image processing device 1 carries out the normalization described above with reference to that information. As another example, when a plurality of cameras having different viewpoints is provided as the camera 51 in the light reflex measurement system 100, the image processing device 1 may recognize the actual size of the pupil size and the interpupillary distance by three-dimensionally restoring the face of the subject 6 from the camera images of the plurality of cameras based on any three-dimensional restoration technique, such as structure from motion (SfM).

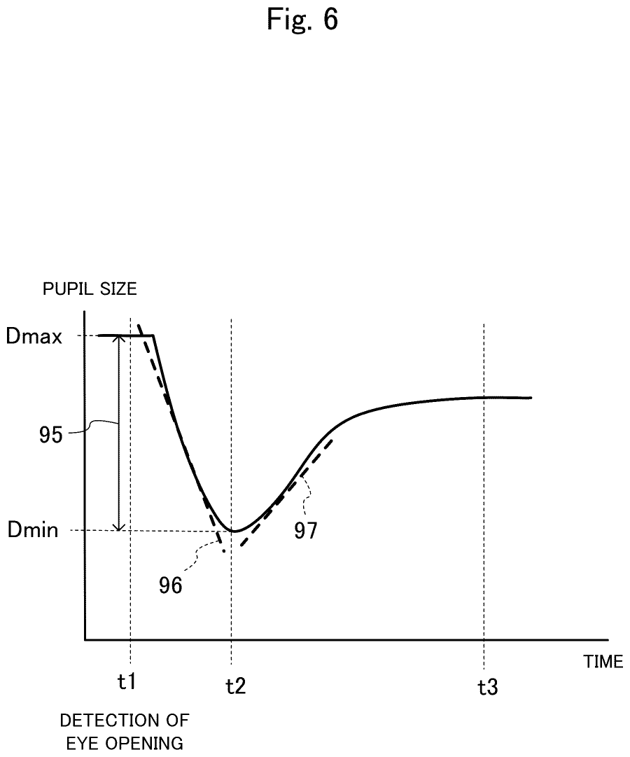

Next, a specific example of the light reflex index will be described. FIG. 6 is an exemplary graph illustrating a temporal change in the pupil size with the light reflex.

In this case, after a time lag (i.e., latency) from time “t1” at which eye opening of the subject 6 based on the eye opening instruction is detected until the pupil starts to contract elapses, the pupil size (pupil radius in this case) of the subject 6 gradually decreases from a maximum value “Dmax”, and becomes a minimum value “Dmin” at time “t2”. Thereafter, the pupil size increases again, and enters a steady state near time “t3”, which is a predetermined time after the time t2.

In this case, for example, the image processing device 1 calculates, as the light reflex index, the minimum value Dmin, the maximum value Dmax, a maximum pupil contraction amount equivalent to a length of an arrow 95 (i.e., Dmax−Dmin), a maximum pupil contraction rate equivalent to a ratio between the maximum value Dmax and the minimum value Dmin, a pupil contraction speed equivalent to an inclination of a line 96, a re-expansion speed equivalent to an inclination of a line 97, and the like. The inclination of the line 96 is equivalent to a decrease rate of the pupil size in a period during which the pupil size decreases, and the inclination of the line 97 is equivalent to an increase rate of the pupil size in a period during which the pupil size increases (re-expands).

The light reflex index is not limited to the index described above, and may be any index based on the time-series pupil size measured in the light reflex estimation period.

### (5) Processing Flow

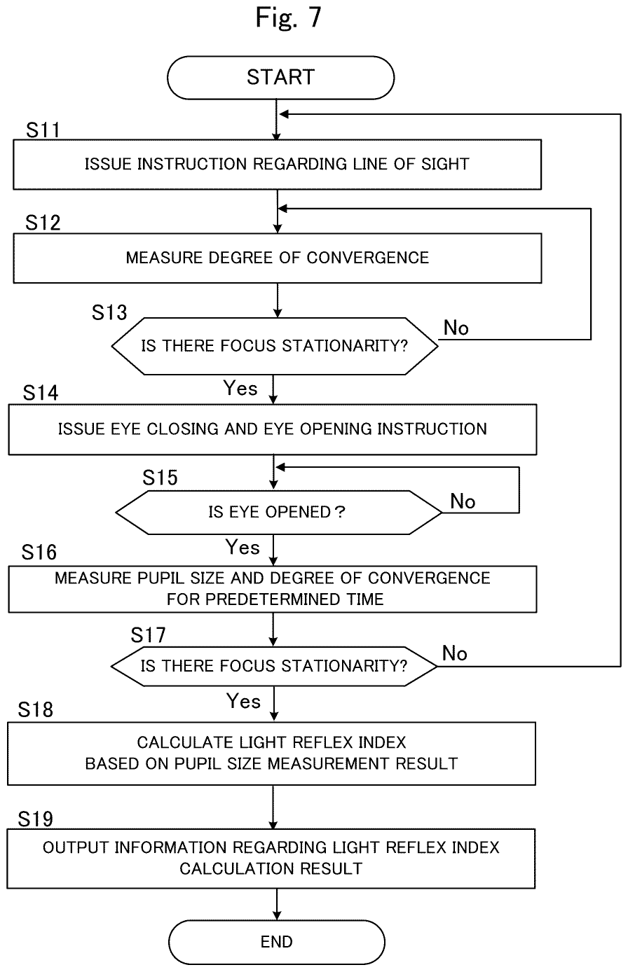

FIG. 7 is an exemplary flowchart regarding the light reflex measurement process to be executed by the image processing device 1. Here, the light reflex measurement process in the case where the pre-closing line-of-sight instruction is issued will be described as an example.

First, the image processing device 1 outputs, using the output device 3, a pre-closing line-of-sight instruction for instructing a line of sight of the subject 6 (step S11). Then, the image processing device 1 measures a degree of convergence of the subject 6 based on each of the time-series camera images generated by the camera 51 after step S11 (step S12).

Then, the image processing device 1 determines whether there is stationarity in the focus of the subject 6 (step S13). In this case, the image processing device 1 determines whether there is stationarity in the focus of the subject 6 based on whether there is a change in the time-series degree of convergence obtained in step S12. Then, if the image processing device 1 determines that there is no stationarity in the focus of the subject 6 (No in step S13), the process returns to step S12, and the degree of convergence is measured.

Then, if it is determined that there is stationarity in the focus of the subject 6 (Yes in step S13), the image processing device 1 outputs, using the output device 3, instructions regarding eye closing and eye opening of the subject 6 (step S14). In this case, for example, the image processing device 1 detects eye closing of the subject 6 after instructing the subject 6 to close the eyes, and then outputs an instruction regarding eye opening after a predetermined time has elapsed.

Next, the image processing device 1 determines whether the subject 6 has opened the eyes (step S15). In this case, for example, the image processing device 1 determines whether the subject 6 has opened the eyes based on the camera image. Then, if it is determined that the subject 6 has not opened the eyes (No in step S15), the image processing device 1 continues to make the determination in step S15.

Then, if it is determined that the subject 6 has opened the eyes (Yes in step S15), the image processing device 1 measures a pupil size and a degree of convergence for a predetermined time based on the latest camera image (step S16). In this case, the image processing device 1 regards the period from when the eye opening of the subject 6 is detected until when a predetermined time elapses as a light reflex estimation period, and continuously measure the pupil size and the degree of convergence based on the camera images generated in the light reflex estimation period. As a result, measurement results (time-series data) of a predetermined number of pupil sizes and degrees of convergence are obtained.

Next, the image processing device 1 determines whether there is stationarity in the focus (step S17). In this case, in a first example, the image processing device 1 determines whether there is stationarity in the focus in the execution period (i.e., light reflex estimation period) of step S16 based on whether there is a change in the time-series degree of convergence obtained in step S16. In a second example, the image processing device 1 makes the determination in step S17 in consideration of the focus before the eye closing in addition to the presence or absence of the stationarity in the focus in the light reflex estimation period. In this case, the image processing device 1 determines whether there is stationarity in the focus based on the degree of convergence obtained in step S12 immediately before the stationarity in the focus is determined in step S13 and the time-series degree of convergence obtained in step S16.

Then, if the image processing device 1 determines that there is no stationarity in the focus (No in step S17), the process returns to step S11. If the image processing device 1 determines in step S17 the stationarity in the focus without considering the measurement result of the degree of convergence in step S12 (i.e., in the case of the first example), the process may return to step S14 instead of returning to step S11.

Then, if it is determined that there is stationarity in the focus (Yes in step S17), one or a plurality of light reflex indexes are calculated based on the measurement result of the time-series pupil size measured in step S16 (step S18). The image processing device 1 may calculate a time average (or another representative value) of the light reflex index based on a plurality of samples of the light reflex index obtained by repeating steps S11 to S18 a plurality of times.

Then, the image processing device 1 outputs, using the output device 3, information regarding the calculation result of the light reflex index (step S19). In this case, the image processing device 1 may determine, from the calculated light reflex index, a state of the subject 6 regarding whether there is an aging phenomenon, whether there is fatigue, whether there is a nervous system disease, and the like depending on the application to be applied, and may output a result of the determination using the output device 3. An example of the application will be described later.

### (6) Modified Example

Next, modified examples of the above-described example embodiment will be described. The following modified examples may be applied to the example embodiment described above in any combination.

### First Modified Example

The image processing device 1 may output, based on the size of the iris of the subject 6 recognized from the camera image, guidance information regarding a shooting distance using the output device 3 in such a way that a distance between the subject 6 and the camera 51 (shooting distance) becomes a predetermined distance.

In this case, for example, the image processing device 1 recognizes the size of the iris of the subject 6 from the camera image before the pre-closing line-of-sight instruction in step S11. Then, the image processing device 1 determines whether the recognized size of the iris belongs to an appropriate size range. The appropriate size range described above is a range of the size of the iris relevant to the range of the shooting distance within a distance range suitable for the light reflex measurement process, and is, for example, a range stored in advance in the storage device 4, the memory 12, or the like. Then, if the recognized size of the iris is out of the appropriate size range, the image processing device 1 causes the output device 3 to output guidance information for notifying the subject 6 of necessity for adjusting the shooting distance. Specifically, the image processing device 1 outputs guidance information suggesting that the shooting distance is shortened if the recognized size of the iris is smaller than the appropriate size range, and outputs guidance information suggesting that the shooting distance is lengthened if the recognized size of the iris is larger than the appropriate size range. Then, if the recognized size of the iris is within the appropriate size range, the image processing device 1 issues the pre-closing line-of-sight instruction in step S11.

According to the present modified example, the image processing device 1 may suitably adjust the shooting distance to be a distance suitable for the measurement of the light reflex.

### Second Modified Example

The image processing device 1 may further execute processing of controlling a light source in such a way that the illuminance of the subject 6 in the light reflex estimation period is constant.

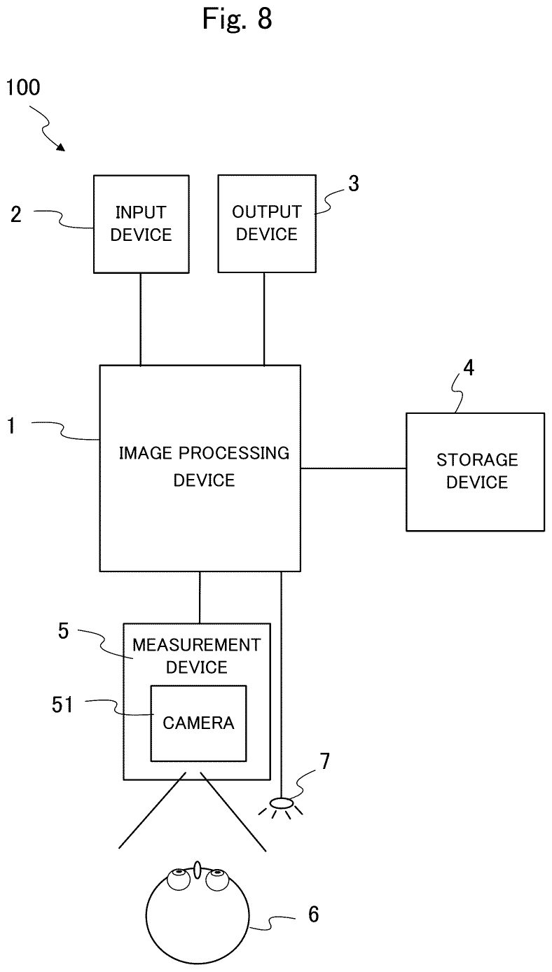

FIG. 8 illustrates a schematic configuration of the light reflex measurement system 100 according to a second modified example. The light reflex measurement system 100 according to the second modified example includes a light source 7. The light source 7 is a light source for illuminating the subject 6, and in a case where the light reflex measurement system 100 is a smartphone or the like as illustrated in FIG. 2, it is a light or a display provided near a camera to be used in the smartphone or the like.

In the present modified example, the image processing device 1 controls a light amount of the light source 7. For example, the image processing device 1 causes the light source 7 to emit light with the same light amount in a period from predetermined timing after the eye closing instruction and before the eye opening instruction to elapse of a predetermined time. In this case, the period described above is set to have a length including at least the entire period of the light reflex estimation period. When the light source 7 is a light, the image processing device 1 may perform on-off control in such a way that the light source 7 is turned on in the period described above and is turned off in a period other than the period described above.

According to the present modified example, the image processing device 1 may control the illuminance of the subject 6 in the light reflex estimation period to be constant, and may suitably suppress a decrease in the light reflex measurement accuracy based on unevenness of the illuminance.

The image processing device 1 may set constancy of the illuminance of the subject 6 in the light reflex estimation period as a condition for measuring the light reflex (i.e., calculating the light reflex index) and making an output, and may measure and output the light reflex only when the illuminance of the subject 6 is stable in the light reflex estimation period. In that case, for example, the image processing device 1 determines whether the illuminance of the subject 6 in the light reflex estimation period is constant based on the time-series illuminance measured by the illuminance sensor of the measurement device 5 in the light reflex estimation period. In that case, for example, the image processing device 1 determines whether the illuminance substantially changes based on a variance of a predetermined number of measured values of the illuminance obtained in the light reflex estimation period, a difference between a maximum value and a minimum value, or comparison between a predetermined threshold and statistics representing other variations. Also in this case, the image processing device 1 may suitably suppress the decrease in the light reflex measurement accuracy based on the unevenness of the illuminance. For example, a luminance value of a camera image may be used instead of the illuminance sensor.

### (7) Application

Next, the application of the light reflex measurement system 100 will be described. In the following application, the light reflex measurement system 100 estimates a state (in particular, state regarding health) of the subject 6 based on the calculated light reflex index, and outputs an estimation result of the state of the subject 6. Here, as a specific example of the application of the light reflex measurement system 100, an application related to a quantitative grasp of a functional decline due to aging and an application related to a quantitative grasp of eyestrain will be exemplified.

### (7-1) Quantitative Grasp of Functional Decline due to Aging

In the application related to the quantitative grasp of a functional decline due to aging, for example, the image processing device 1 calculates, as light reflex indexes, a pupil size measured immediately after the eye opening (i.e., maximum value Dmax of the pupil size), a pupil size when the light reflex occurs after the eye opening (i.e., minimum value Dmin of the pupil size or pupil size after re-expansion), a pupil contraction speed, and the like. Then, the image processing device 1 compares the calculated values of the various calculated light reflex indexes with reference values of the light reflex indexes, and quantitatively estimates a degree of the functional decline due to aging of the subject 6. Then, the image processing device 1 outputs a result of the estimation to the output device 3. The reference values described above may be criteria values of various general light reflex indexes at the age of the subject 6, or may be past calculated values of the calculated values of the light reflex indexes of the subject 6. The reference values described above may be a threshold for determining presence or absence or a level of the functional decline due to aging.

Here, the image processing device 1 may use a model for estimating a degree of the functional decline due to aging of the subject 6, and may cause the output device 3 to output information output by the model. The model described above is a machine learning model or the like such as a formula, a lookup table, or a neural network, and for example, outputs an estimation result (e.g., estimated age of the subject 6, etc.) regarding the degree of the functional decline due to aging when calculated values of various light reflex indexes (or differences between the calculated values and reference values, etc.) are input. Parameters and the like of the model described above are stored in advance in the storage device 4, the memory 12, or the like. The image processing device 1 may display a graph or the like showing the calculated values of the various calculated light reflex indexes and the reference values relevant thereto in a comparable manner.

According to such an application, the subject 6 is enabled to easily measure a light reflex by the subject 6 him/herself using his/her own smartphone or the like, and to quantitatively grasp a functional decline due to aging. In addition, the image processing device 1 may propose an activity for preventing the functional decline due to aging, or may visualize the effect of prevention of the functional decline due to aging, which may enhance the awareness of the user, who is the subject 6, regarding continuation of the activity for preventing the functional decline due to aging.

### (7-2) Quantitative Grasp of Eyestrain

In the application related to the quantitative grasp of eyestrain, the image processing device 1 calculates, for example, a maximum pupil contraction rate, a pupil contraction speed, a re-expansion speed, and the like as light reflex indexes. Then, the image processing device 1 compares the calculated values of the various calculated light reflex indexes with reference values of the light reflex indexes, and quantitatively estimates a degree of the eyestrain of the subject 6. Then, the image processing device 1 outputs a result of the estimation to the output device 3. The reference values described above may be criteria values of various general light reflex indexes at the age of the subject 6, or may be past calculated values of the light reflex indexes of the subject 6. The reference values described above may be a threshold for determining presence or absence or a level of the eyestrain of the subject 6.

Here, the image processing device 1 may use a model for estimating a degree of the eyestrain of the subject 6, and may cause the output device 3 to output information output by the model. The model described above is a machine learning model or the like such as a formula, a lookup table, or a neural network, and for example, outputs an estimation result regarding the degree of the eyestrain of the subject 6 when calculated values of various light reflex indexes (or differences between the calculated values and reference values, etc.) are input. Parameters and the like of the model described above are stored in advance in the storage device 4, the memory 12, or the like. The image processing device 1 may display, as information indicating the degree of the eyestrain of the subject 6, a graph or the like showing the calculated values of the various calculated light reflex indexes and the reference values relevant thereto in a comparable manner.

According to such an application, the subject 6 is enabled to easily measure a light reflex by the subject 6 him/herself using his/her own smartphone or the like, and to quantitatively grasp eyestrain.

### Second Example Embodiment

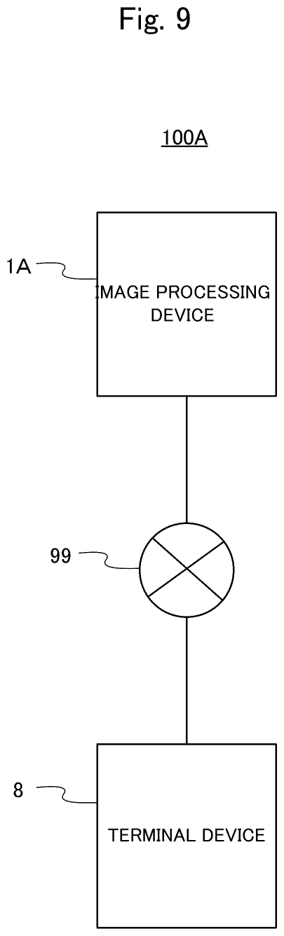

FIG. 9 illustrates a schematic configuration of a light reflex measurement system 100A according to a second example embodiment. The light reflex measurement system 100A according to the second example embodiment includes an image processing device 1A, which functions as a server, and a terminal device 8, which is used by a subject and functions as a client. The image processing device 1A and the terminal device 8 perform data communication via a network 99. Hereinafter, the same components as those of the first example embodiment will be appropriately denoted by the same reference signs, and descriptions thereof will be omitted.

The terminal device 8 is a terminal to be used by a user as the subject, and has an input function, a display function, a communication function, and an imaging function to function as the input device 2, the output device 3, the measurement device 5 including the camera 51, and the like illustrated in FIG. 1. The terminal device 8 may be, for example, a personal computer, a tablet terminal such as a smartphone, a personal digital assistant (PDA), or the like. The terminal device 8 transmits a facial image of the subject output from a camera 51 to the image processing device 1A via the network 99.

The image processing device 1A has the same hardware configuration as the hardware configuration of the image processing device 1 illustrated in FIG. 2, and a processor 11 of the image processing device 1A has the functional blocks illustrated in FIG. 4 described in the first example embodiment. Then, the image processing device 1A receives a camera image from the terminal device 8 via the network 99, and executes a light reflex measurement process for the subject. The image processing device 1A transmits an output signal for outputting a processing result to the terminal device 8 via the network 99 based on a display request from the terminal device 8.

As described above, the image processing device 1A according to the second example embodiment may perform processing related to measurement of a light reflex of the subject, who is the user of the terminal device 8, and may suitably present a result of the measurement of the light reflex to the subject using the terminal device 8.

### Third Example Embodiment

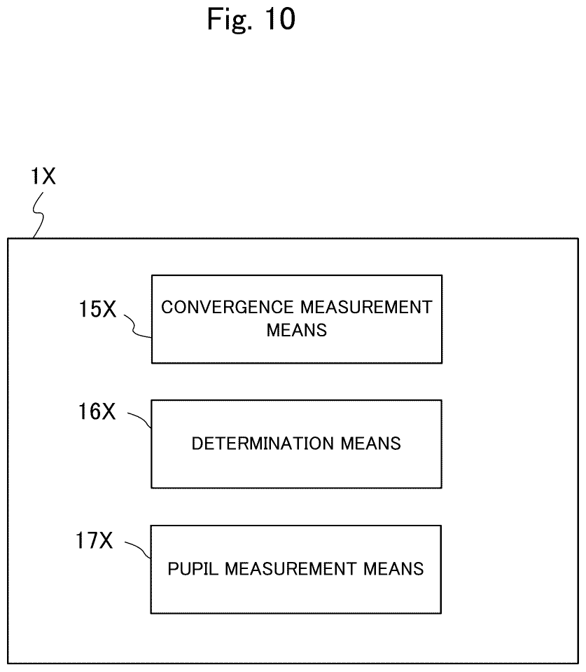

FIG. 10 illustrates a block diagram of the image processing device 1X according to the third example embodiment. The image processing device 1X mainly includes a convergence measurement means 15X, a determination means 16X, and a pupil measurement means 17X. The image processing device 1X may be configured by plural devices.

The convergence measurement means 15X is configured to measure a degree of convergence of a subject after eye opening, based on an image obtained by capturing the subject who has performed the eye opening after eye closing. Examples of the convergence measurement means 15X include the convergence measurement unit 15 according to the first or second example embodiment.

The determination means 16X is configured to determine presence or absence of a focus change of the subject, based on the degree of convergence. Examples of the determination means 16X include the focus change determination unit 16 according to the first or second example embodiment.

The pupil measurement means 17X is configured to measure a pupil of the subject based on the image obtained by capturing the subject, upon determining the absence of the focus change. Examples of the pupil measurement means 17X include pupil size measurement unit 17 according to the first or second example embodiment.

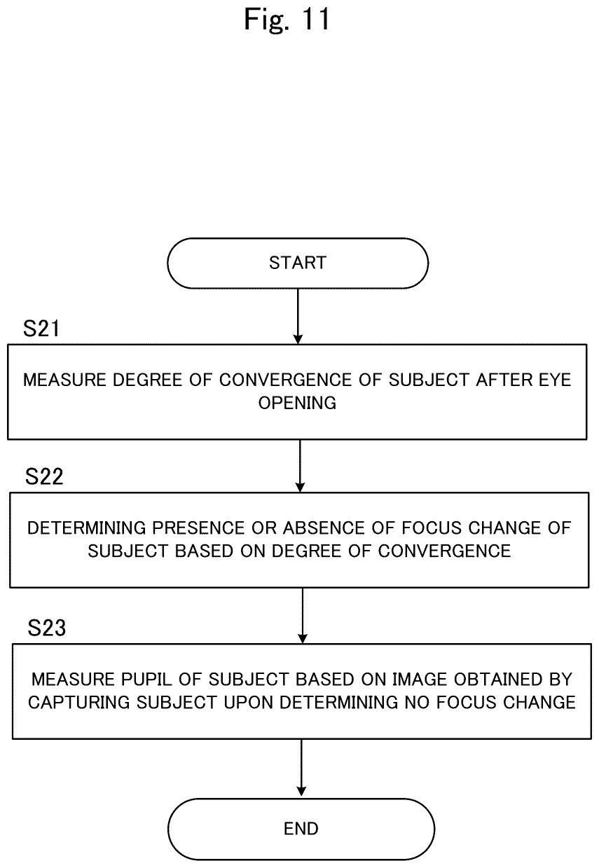

FIG. 11 illustrates a flowchart executed by the image processing device 1X according to the third example embodiment. First, the convergence measurement means 15X measures a degree of convergence of a subject after eye opening, based on an image obtained by capturing the subject who has performed the eye opening after eye closing (step S21). Then, the determination means 16X determines presence or absence of a focus change of the subject, based on the degree of convergence (step S22). The pupil measurement means 17X measures a pupil of the subject based on the image obtained by capturing the subject, upon determining the absence of the focus change (step S23).

The image processing device 1X according to the third example embodiment can suitably measure the pupil of the subject along with the light reflex.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### Supplementary Note 1

An Image Processing Device Comprising:
a convergence measurement means for measuring a degree of convergence of a subject after eye opening, based on an image obtained by capturing the subject who has performed the eye opening after eye closing;
a determination means for determining presence or absence of a focus change of the subject, based on the degree of convergence; and
a pupil measurement means for measuring a pupil of the subject based on the image obtained by capturing the subject, upon determining the absence of the focus change.

### Supplementary Note 2

The image processing device according to Supplementary Note 1, further comprising a light reflex calculation means for calculating an index regarding a light reflex of the subject based on time-series measurement results of the pupil.

### Supplementary Note 3

The image processing device according to Supplementary Note 1, wherein
the determination means determines the presence or absence of the focus change in a predetermined period based on the image obtained by capturing the subject in the predetermined period after the eye opening, and
the pupil measurement means measures the pupil based on the image obtained by capturing the subject in the predetermined period, upon determining the absence of the focus change in the predetermined period.

### Supplementary Note 4

The image processing device according to Supplementary Note 3, wherein
the determination means further determines the presence or absence of the focus change before the eye closing and after the eye opening, and
the pupil measurement means measures the pupil based on the image obtained by capturing the subject in the predetermined period, upon determining the absence of the focus change in the predetermined period and the absence of the focus change before the eye closing and after the eye opening.

### Supplementary Note 5

The image processing device according to Supplementary Note 3, further comprising
a light source control means for controlling a light amount in such a way that the light amount of a light source that illuminates the subject is constant in the predetermined period.

### Supplementary Note 6

The image processing device according to Supplementary Note 1, wherein
the image includes an image obtained by capturing at least both eyes of the subject, and
the convergence measurement means calculates the degree of convergence based on a position of the pupil of the eyes in the image.

### Supplementary Note 7

The image processing device according to Supplementary Note 1, further comprising
a guidance information output means for outputting, using an output device, guidance information regarding a distance between the subject and an imaging means for generating the image, based on a measurement result obtained by measuring an iris of the subject based on the image.

### Supplementary Note 8

The image processing device according to Supplementary Note 1, further comprising an instruction means for instructing the subject to perform the eye closing and the eye opening after elapse of a predetermined time from the eye closing.

The image processing device according to Supplementary Note 8,
wherein the instruction means issues an instruction regarding a line of sight of the subject, and, upon determining the absence of the focus change of the subject after the instruction, instructs the subject to perform the eye closing.
   supplementary Note 10

The image processing device according to Supplementary Note 1,
wherein the pupil measurement means measures a size of the pupil.

### Supplementary Note 11

The image processing device according to Supplementary Note 2,
wherein the light reflex calculation means outputs, using an output device, information regarding a state of the subject estimated based on the index.

### Supplementary Note 12

The image processing device according to Supplementary Note 11,
wherein the light reflex calculation means outputs, as the information regarding the state, a degree of a functional decline due to aging of the subject estimated based on the index.
   supplementary Note 13

The image processing device according to Supplementary Note 11,wherein the light reflex output means outputs, as the information regarding the state, a degree of eyestrain of the subject estimated based on the index.

supplementary Note 14

An image processing method to be executed by a computer, the method comprising: measuring a degree of convergence of a subject after eye opening, based on an image obtained by capturing the subject who has performed the eye opening after eye closing;
determining presence or absence of a focus change of the subject, based on the degree of convergence; and
measuring a pupil of the subject based on the image obtained by capturing the subject, upon determining the absence of the focus change.

### Supplementary Note 15

A storage medium storing a program for causing a computer to perform a process comprising:
measuring a degree of convergence of a subject after eye opening, based on an image obtained by capturing the subject who has performed the eye opening after eye closing;
determining presence or absence of a focus change of the subject, based on the degree of convergence; and
measuring a pupil of the subject based on the image obtained by capturing the subject, upon determining the absence of the focus change.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1A, 1X Image processing device
2 Input device
3 Output device
4 Storage device
5 Measurement device
7 Light source
8 Terminal device
11 Processor
12 Memory
13 Interface
51 Camera
90 Data bus
99 Network
100,100A Light reflex measurement system

What is claimed is:

1. An image processing device comprising:
at least one memory configured to store instructions; and
at least one processor configured to execute the instructions to:
measure a degree of convergence of a subject after eye opening, based on an image obtained by capturing the subject who has performed the eye opening after eye closing;
determine presence or absence of a focus change of the subject, based on the degree of convergence; and
measure a pupil of the subject based on the image obtained by capturing the subject, upon determining the absence of the focus change.

2. The image processing device according to claim 1,
wherein the at least one processor configured to execute the instructions to calculate a index regarding a light reflex of the subject based on time-series measurement results of the pupil.

3. The image processing device according to claim 1, wherein
the at least one processor is configured to execute the instructions to determine the presence or absence of the focus change in a predetermined period based on the image obtained by capturing the subject in the predetermined period after the eye opening, and
the at least one processor is configured to execute the instructions to measure the pupil based on the image obtained by capturing the subject in the predetermined period, upon determining the absence of the focus change in the predetermined period.

4. The image processing device according to claim 3, wherein
the at least one processor is configured to execute the instructions to further determine the presence or absence of the focus change before the eye closing and after the eye opening, and
the at least one processor is configured to execute the instructions to measure pupil measurement means measures the pupil based on the image obtained by capturing the subject in the predetermined period, upon determining the absence of the focus change in the predetermined period and the absence of the focus change before the eye closing and after the eye opening.

5. The image processing device according to claim 3, further
wherein at least one processor is configured to execute the instructions to control light amount in such a way that the light amount of a light source that illuminates the subject is constant in the predetermined period.

6. The image processing device according to claim 1, wherein
the image includes an image obtained by capturing at least both eyes of the subject, and
the at least one processor is configured to execute the instructions to calculate the degree of convergence based on a position of the pupil of the eyes in the image.

7. The image processing device according to claim 1,
wherein at least one processor is configured to execute the instructions to output using an output device, guidance information regarding a distance between the subject and an imaging means for generating the image, based on a measurement result obtained by measuring an iris of the subject based on the image.

8. The image processing device according to claim 1,
wherein at least one processor is configured to execute the instructions to instruct the subject to perform the eye closing and the eye opening after elapse of a predetermined time from the eye closing.

9. The image processing device according to claim 8,
wherein the at least one processor is configured to execute the instructions to issue an instruction regarding a line of sight of the subject, and, upon determining the absence of the focus change of the subject after the instruction, instructs the subject to perform the eye closing.

10. The image processing device according to claim 1,
wherein the at least one processor is configured to execute the instructions to measure a size of the pupil.

11. The image processing device according to claim 2,
wherein the at least one processor is configured to execute the instructions to output, using an output device, information regarding a state of the subject estimated based on the index.

12. The image processing device according to claim 11,
wherein the at least one processor is configured to execute the instructions to output, as the information regarding the state, a degree of a functional decline due to aging of the subject estimated based on the index.

13. The image processing device according to claim 11,
wherein the at least one processor is configured to execute the instructions to output, as the information regarding the state, a degree of eyestrain of the subject estimated based on the index.

14. An image processing method to be executed by a computer, the method comprising:
measuring a degree of convergence of a subject after eye opening, based on an image obtained by capturing the subject who has performed the eye opening after eye closing;
determining presence or absence of a focus change of the subject, based on the degree of convergence; and
measuring a pupil of the subject based on the image obtained by capturing the subject, upon determining the absence of the focus change.

15. A non-transitory computer readable storage medium storing a program for causing a computer to perform a process comprising:
measuring a degree of convergence of a subject after eye opening, based on an image obtained by capturing the subject who has performed the eye opening after eye closing;
determining presence or absence of a focus change of the subject, based on the degree of convergence; and
measuring a pupil of the subject based on the image obtained by capturing the subject, upon determining the absence of the focus change.
